(19) United States
(12) Patent Application Publication
(10) Pub. No.: US 2003/0099616 A1
(43) Pub. Date: May 29, 2003

(54) Dual specificity tumor killing vectors driven by the telomerase promoter

(76) Inventors: John M. Irving, San Mateo, CA (US); David B. Karpf, Monte Sereno, CA (US); J. Michael Schiff, Menlo Park, CA (US)

Correspondence Address: GERON CORPORATION, 230 CONSTITUTION DRIVE, MENLO PARK, CA, 94025

(21) Appl. No.: 10/206,447

(22) Filed: Jul. 25, 2002

Related U.S. Application Data
(60) Provisional application No. 60/308,029, filed on Jul. 25, 2001.

(51) Int. Cl. A61K 48/00; C12N 7/00; C12N 15/861; C12N 7/01

(52) U.S. Cl. 424/93.2; 435/320.1; 435/456; 435/235.1

(57) ABSTRACT

It has been discovered that the specificity of multiple transcriptional regulatory elements can be combined to make vector systems that selectively target cancer cells. The promoter for telomerase reverse transcriptase (TERT) can be combined in a remarkably synergistic fashion with another promoter that has expression restricted to cancer cells or a particular tissue type. The two promoters work synergistically for exquisite targeting of the malignant cells—where it causes cell lysis while leaving neighboring healthy cells intact.

### REFERENCE TO RELATED APPLICATIONS

This application claims priority to U.S. provisional application 60/308,029, filed Jul. 25, 2001. The priority application is hereby incorporated herein by reference in its entirety, along with U.S. Pat. Application Ser. Nos. 09/615,039 and 60/256,418.

### TECHNICAL FIELD

This invention relates generally to the fields of virology, gene therapy, and telomere biology. More specifically, the disclosure provides viruses comprising multiple heterologous replication elements in a viral construct for specific killing of cancer cells.

### BACKGROUND

Many forms of cancer are intractable to traditional courses of radiation or small molecule pharmaceuticals. Considerable interest has evolved in developing gene therapy vectors as therapeutic agents.

A broad variety of therapeutic genes are currently under investigation in preclinical and in clinical studies (reviewed by Walther et al., Mol. Biotechnol. 13:21, 1999). The candidate genes have different origins and mechanisms of action, such as cytokine genes, genes coding for immunostimulatory molecules/antigens, genes encoding prodrug-activating enzymes (suicide genes), genes that promote apoptosis, and tumor suppressor genes.

Vectors for delivering such genes can be based on viral and non-viral systems. For example, viral vectors can be based on herpes family viruses. U.S. Pat. No. 5,728,379 (Georgetown University) relates to replication competent HSV containing a transcriptional regulatory sequence operatively linked to an essential HSV gene. U.S. Pat. No. 6,139,834 reports that replication-competent herpes simplex virus mediates destruction of neoplastic cells. U.S. Pat. No. 5,997,859 and EP 702084 B1 (Chiron) pertain to replication-detective recombinant retrovirus, carrying a vector construct capable of preventing, inhibiting, stabilizing or reversing infections, cancer, or autoimmune disease. WO 99/08692 proposes the use of reovirus in treating cancer, particularly ras-mediated neoplasms.

Many proposed cancer therapeutic vectors are based on adenovirus. U.S. Pat. Nos. 5,631,236 and 6,096,718 (Baylor College of Medicine) cover a method of causing regression in a solid tumor, using a vector containing an HSV thymidine kinase (tk) gene, followed by administration of a prodrug such as ganciclovir. U.S. Pat. No. 6,096,718 (Baylor College of Medicine) relates to the use of a replication incompetent adenoviral vector, comprising an HSV tk gene under control of the α-lactalbumin promoter.

U.S. Pat. Nos. 5,801,029 and 5,846,945 (Onyx Pharmaceuticals) relate to adenovirus in which the E1b gene has been altered so as not to bind and inactivate tumor suppressor p53 or RB proteins expressed by the host. This prevents the virus from inactivating tumor suppression in normal cells, which means the virus cannot replicate. However, the virus will replicate and lyse cells that have shut off p53 or RB expression through oncogenic transformation.

U.S. Pat. No. 5,998,205 and WO 99/25860 (GTI/Novartis) pertain to a tissue-specific replication-conditional adenovirus, comprising a transcriptional regulatory sequence (such as the α-fetoprotein promoter) operably linked to an adenovirus early replication gene. U.S. Pat. No. 5,698,443 and WO 98/39464 (Calydon) provides replication-conditional adenoviruses controlled by the PSA promoter. Yu et al. (Cancer Res. 59:1498, 1999) identified the transcriptional regulatory sequences of human kallikrein 2, and used them to construct an attenuated replication competent adenovirus designated Calydon Virus 764 for prostate cancer therapy. WO 96/34969 (Canji) outlines a method for treating mammalian cancer cells with a replication competent adenoviral vector containing a therapeutic gene and a disease-specific regulatory region linked to a replication gene of the vector.

Alemany et al. (Cancer Gene Ther. 6:21, 1999) outline complementary adenoviral vectors for oncolysis. One vector contains cis replication elements and E1 a under control of a tissue-specific promoter. The supplemental vector contains all other trans-acting adenovirus replication genes. Coinfection leads to controlled killing of hepatocarcinoma cells. Hernandez-Alcoceba et al. (Hum. Gene Ther. 11:2009, 2000) report construction and testing of a conditionally replicative adenovirus in which the E1a and E4 promoters have been replaced by a portion of the pS2 promoter containing two estrogen-responsive elements. The vector was able to complement E1a function in trans for a conventional E1a-deleted adenovirus, and was designed for treatment of breast cancer.

International Patent Publication WO 98/14593 (Geron) describes an adenovirus construct in which the tk gene is placed under control of the promoter for telomerase reverse transcriptase (TERT). This gene is expressed at high levels in cancer cells of any tissue type, and the vector renders cancer cell lines susceptible to toxic effects of ganciclovir. WO 00/46355 (Geron) describes an oncolytic virus having a genome in which a TERT promoter is linked to a genetic element essential for replication or assembly of the virus, wherein replication of the virus in a cancer cell leads to lysis of the cancer cell.

Koga et al. (Hu. Gene Ther. 11:1397, 2000) proposed a telomerase-specific gene therapy using the human TERT gene promoter linked to the apoptosis gene Caspase-8 (FLICE). Shahrokh et al. (Cancer Res. 61:2562, 2001) tested adenovirus vectors expressing conditional Caspase-1 and Caspase-3 in a gene therapeutic approach to prostate cancer. Gu et al. (Cancer Res. 60:5359, 2000) reported a binary adenoviral system that induced Bax expression via the hTERT promoter. They found that it elicited tumor-specific apoptosis in vitro and suppressed tumor growth in nude mice.

There is a need to develop new constructs with improved safety and efficacy for use in cancer therapy.

### SUMMARY

This disclosure provides a system for gene therapy of target tissues using particles made from a viral genome and heterologous genetic elements. Cell-killing vectors can be produced that target particular tissue types, and are suitable for use in cancer therapy. Featured vector particles couple the specificity of transcription control elements for telomerase and a tissue or tumor specific gene, which bestows the vector with a high degree of specificity for malignant cells of a particular tissue type.

One embodiment of the invention is a vector comprising two genes each under control of a different heterologous transcriptional control element, one of which preferentially promotes transcription in cells expressing telomerase reverse transcriptase (TERT). Vectors falling within this embodiment may optionally comprise other genes sharing one of these control elements, or under control of further heterologous control elements. Included is a viral vector having a gene under control of a transcriptional control element for a telomerase reverse transcriptase (TERT), and another gene under control of a heterologous transcriptional control element for a tissue or tumor specific gene other than TERT.

The gene controlled by one or both of these heterologous transcriptional control elements may be a gene required for replication or assembly of the vector, in which case the vector is replication-conditional, and may effect killing of the host cell in the course of the replication cycle. Exemplary constructs are made using genes from an adenovirus E1a, E1b, E2, or E4 region, the ICP0 or ICP4 genes of herpes virus, or other genes of viral or non-viral origin that can act to functionally replace vector replication genes (such as the immediate early genes of CMV, or a Y-box transactivator).

Exemplary TERT control elements are taken from the human TERT gene upstream sequence, or share homology with the human sequence. The other heterologous transcriptional control element is exemplified by tissue and tumor specific promoters or enhancers, such as the promoter for telomerase RNA component, and other promoters listed later in this disclosure.

The vector may further comprise an encoding region for an effector gene, controlled by one of the control elements already referred to, or by its own specific or constitutively expressed control element. The effector region can cause or contribute to direct killing of the host cell, or initiate a process that prevents the cell from its usual course of replication or malignant transformation. Exemplary effector genes are directly toxic to the cell, render the cell more susceptible to toxic effects of a drug, express a cytokine, or have a tumor suppressor function. Also exemplary are ribozymes, RNAi, or complementary sequences that interfere with hTERT translation or telomerase activity.

When transduced into human or other mammalian cells expressing TERT (including most cancer cells), the vectors of this invention can cause killing of the cell or its progeny directly or indirectly in any of a number of different ways. For example, the vector may be directly lethal to the cell, either by replicating in the cell, or by expressing a toxic gene. Alternatively, the vector may cause killing of the cell or its progeny by limiting telomerase activity, by rendering the cell susceptible to the toxic effects of another drug or antibody, by otherwise compromising cell viability or replicative capacity, or by otherwise rendering them more susceptible to other chemotherapeutic agents included in the treatment protocol.

This invention also includes methods for selecting vectors such as those already described, in which host cells are transduced with a vector construct, and the host cell is monitored for any effect of the vector. The vectors of this invention can be formulated in a medicament and used for treating a condition associated with increased expression of telomerase reverse transcriptase in affected cells, such as cancer.

Other embodiments of the invention will be apparent from the description that follows.

### DRAWINGS

FIG. 1 is a half tone reproduction of cell lines photographed 7 days after infection with oncolytic virus. Top row: uninfected cells (negative control). Middle row: cells infected with oncolytic adenovirus, in which replication gene E1a is operably linked to the hTERT promoter. Bottom row: cells infected with adenovirus in which E1a is operably linked to the CMV promoter (positive control).

The cells tested were as follows: Top panels: BJ (foreskin fibroblast); IMR-90 (lung fibroblast); WI-38 (lung fibroblast); cells of non-malignant origin. Bottom panels: DAOY (medulloblastoma); HeLa (cervical carcinoma); HT1080 (fibrosarcoma). The results show that the hTERT-regulated oncolytic virus (AdhTERTpE1a) specifically lyses cancer cells, in preference to cell lines that don't express telomerase reverse transcriptase at a substantial level. This is in contrast to oncolytic virus regulated by a constitutive promoter like CMV promoter (AdCMVpE1), which lyses both normal and malignant cells non-specifically.

FIG. 2 is a graph showing the effect of an adenovirus driven by the hTERT promoter on osteosarcoma tumors in a mouse model. The group labeled “Ad5Emp” is the vector control, with tumors growing to 400 mm³ within a month of injection. The groups labeled “Onco2” were injected intratumorally with the replication-conditional vector on days 11-15, and showed considerable reduction in tumor growth rate.

### DETAILED DESCRIPTION

A promising therapeutic approach to cancer is to target tumors with a virus that replicates specifically in cancer cells, destroying them in the process. Replication conditional adenovirus and herpesvirus constructs have been made and are currently being tested in human clinical trials.

However, the use of viral vectors for human therapy has significant risks—as illustrated by the recent tragedy in the ornithine transcarbamylase gene therapy trial conducted at the University of Pennsylvania (M. Balter, Science 288:951, 2000). Replication competent adenovirus increases the degree of concern, since efficacy in cancer cell killing ensues in part from an ability of the vector to transmit from cell to cell.

It was known previously that the lytic specificity of a virus that replicates under control of a single tissue specific promoter is insufficient for systemic administration. For example, replication conditional adenovirus in which the E1a region is driven by the PSA promoter shows specificity for prostate cancer cells of about 1:100 (Yu et al., Cancer Res. 59:1498, 1999). This means that the use of viruses controlled by a single tumor-specific promoter can be limited to direct intratumoral administration.

The invention described in this disclosure embraces the discovery that the specificity of a tissue specific promoter can be coupled with a telomerase promoter for exquisite targeting of cancer cells. Telomerase reverse transcriptase (TERT) is the catalytic subunit of the enzyme that prevents telomere loss in replicating cells, thus extending their replicative capacity (Harley et al., Cancer Surv. 29:263, 1997). It is expressed constitutively in embryonic stem cells, but shuts down after differentiation into particular cell types. Virtually all truly immortal cancer cells reactivate TERT in order to maintain replication potential, which means that the TERT promoter can be used to drive transcription in almost all life threatening cancers.

It has been found that the TERT promoter can be combined in a remarkably synergistic fashion with a promoter that has expression restricted to a particular tissue type. By coupling properties of two promoters with different selectivity, a vector can be created that is both tissue specific, and specific for malignant cells. In this way, the vector is targeted with extreme precision to the disease site—where it causes cell lysis while leaving neighboring healthy cells intact.

This provides the following important advantages:

Ability to target more tumor types. Oncolytic viruses targeted using a tissue specific transcriptional control element alone (like the PSA enhancer or the α-fetoprotein promoter) rely on the fact that the control element is expressed in cells of a particular tissue type with some prominence in cells that are malignant. There are many common cancer types where promoters with this type of specificity have not been identified. This invention overcomes the limitation, because the TERT promoter can be teamed up with any tissue specific promoter that is specifically upregulated in the target tissue type, regardless of whether it is preferentially expressed in tumor cells. Alternatively, the TERT promoter can be teamed up with another tumor-specific promoter, like the promoter for telomerase RNA component—thereby creating a universal tumor killing vector. Essentially all malignant tumors should be amenable to treatment using the vectors of this invention.

Improved safety. By controlling the effector function of the virus by two unrelated specificities, there is a synergistic margin of safety, minimizing expression in other tissue types and non-malignant cells of the same tissue type. For example, a vector controlled by the TERT and albumin promoters given intravenously would pose virtually no threat to non-malignant liver cells (which don't express TERT), and non-hepatocytes outside the liver—even bone marrow stem cells (some of which may express TERT, but none of which express albumin). The vector would specifically target and kill hepatocytes co-expressing albumin and TERT (i.e., hepatocarcinoma cells).

Ability to administer the therapeutic agent systemically. Use of a single tissue- or tumor-specific promoter typically provides a specificity of expression that is about 100-fold in target tissues in comparison with other tissue types. This is generally inadequate for systemic administration, meaning that the use of the construct is limited to intratumoral injection. However, combining a tissue- or tumor-specific promoter or effector with the TERT promoter is expected in some instances of providing a specificity of at least 1 in 10⁴. Because of the synergistic specificity, the therapeutic vectors of this invention can be administered systemically—which considerably enhances the ease of administration, the expected efficacy, and the range of tumors amenable for therapy.

Management of metastatic cancer. Administering the vectors of this invention systemically makes them available for surveillance of metastatic tumor cells proliferating outside the primary site. Metastatic cells will continue to express TERT in order to maintain their proliferative capacity; and dual control of the vector will continue to ensure that only the metastases (not the surrounding tissue) will be subject to the effects of the vector. In this way, the patient will receive simultaneous treatment of the primary tumor and any metastatic disease by way of each therapeutic administration.

Further advantages of the compositions and procedures of this invention are described in the sections that follow.

Definitions

The term “polynucleotide” refers to a polymeric form of nucleotides of any length. Included are genes and gene fragments, mRNA, cDNA, recombinant polynucleotides, branched polynucleotides, plasmids, viral and non-viral vectors and particles, nucleic acid probes, amplification primers, and their chemical equivalents. As used in this disclosure, the term polynucleotide refers interchangeably to double- and single-stranded molecules. Unless otherwise specified, any embodiment of the invention that is a polynucleotide encompasses both a double-stranded form, and each of the two complementary single-stranded forms known or predicted to make up the double-stranded form.

A cell is said to be “genetically altered”, “transfected”, or “genetically transformed” when a polynucleotide has been transferred into the cell by any suitable means of artificial manipulation, or where the cell is a progeny of the originally altered cell that has inherited the polynucleotide.

A “control element” or “control sequence” is a nucleotide sequence that contributes to the functional regulation of a polynucleotide, such as replication, duplication, transcription, splicing, translation, or degradation of a polynucleotide. Transcriptional control elements include promoters, enhancers, and repressors.

Recitation of particular transcriptional control elements, like those for TERT and PSA, refer to polynucleotide sequences derived from the gene referred to that control transcription of an operatively linked gene expression product. It is recognized that various portions of the upstream and intron untranslated gene sequence may in some instances contribute to transcription control, and that all or any subset of these portions may be present in the genetically engineered construct referred to. The control element may be based on the gene sequence of any species having the gene, unless explicitly restricted, and may incorporate any additions, substitutions or deletions desirable, as long as the ability to promote transcription in the target tissue is maintained. Unless otherwise specified, a promoter identified by its gene of origin (such as a TERT promoter) will contain a segment that is at least 90% identical to a sequence taken from within or in front of the gene referred to, and capable of driving transcription in cells where the gene is normally expressed. A particular transcription control sequence can be tested for activity and specificity, for example, by operatively linking to a reporter gene (Example 1).

Numbering of genes, transcriptional control elements, or other features as “first” or “second” may be made in certain places in this disclosure when it is desirable to refer back to these features. The numbering is arbitrary and made for convenience only; it does not imply any particular order in a genetic construct or any ranking in functional importance.

When comparison is made between polynucleotides for degree of identity, it is implicitly understood that complementary strands are easily generated, and the sense or antisense strand is selected or predicted that maximizes the degree of identity between the polynucleotides being compared. Percentage of sequence identity is calculated by first aligning the polynucleotide being examined with the reference counterpart, and then counting the number of residues shared between the sequences being compared as a percentage of the region under examination, without penalty for the presence of obvious insertions or deletions.

“Stringent hybridization conditions” are conditions under which a probe will specifically hybridize to its target sequence but not to other sequences. Generally, stringent conditions are about 5-10° C. lower than the thermal melting point (Tₘ) for the specific sequence at a defined ionic strength and pH. Typical conditions of high stringency for the binding of a probe of about 100 base pairs and above is a hybridization reaction at 65° C. in 2×SSC, followed by repeat washes at 0.1×SSC—or the equivalent combination of solvent and temperature conditions for the particular nucleic acids being studied.

Genetic elements are said to be “operatively linked” if they are in a structural relationship permitting them to operate in a manner according to their expected function. For instance, if a promoter initiates transcription of a coding sequence, the coding sequence can be referred to as operatively linked to (or under control of) the promoter. There may be intervening sequence between the promoter and coding region so long as this functional relationship is maintained.

In the context of encoding sequences, promoters, and other gene elements, the term “heterologous” indicates that the element is derived from a genotypically distinct entity from that of the rest of the entity to which it is being compared. For example, a promoter or gene introduced by genetic engineering techniques into a context in which it does not occur in nature is said to be a heterologous polynucleotide. A heterologous gene or heterologous promoter in an adenovirus vector is a genetic element derived from another virus, a genetic element derived from a prokaryote or eukaryote, or an artificial sequence unrelated to adenovirus. An “endogenous” genetic element is an element that is in the same place in the chromosome or viral genome where it occurs in nature, although other gene elements may be artificially introduced into a neighboring position.

A “replication-conditional” virus comprises a gene essential for replication or assembly of the virus that is preferentially transcribed in cells of a certain type, compared with other cells of the same species. Viruses can be made replication-conditional by placing a gene required for replication or assembly under control of a transcriptional control element that activates or derepresses transcription in certain cell types, compared with others. Exemplary transcriptional control elements are listed later in this disclosure.

A “cytolytic virus” is a virus that lyses or kills a host cell by replicating in the cell, thereby causing the cell to rupture. An “oncolytic virus” is a cytolytic virus that is replication-conditional for cancer cells (and possibly other cell types). It is understood that such viruses are not confined to use with cancer cells, and can be used in vitro or in vivo for any desirable purpose.

A “replication gene” in a vector is a gene that encodes a transcript whose expression is necessary for replication of the vector (usually in combination with other replication genes). The role a particular replication gene plays in the replication process can be anything needed for replication to occur, such as synthesis of genetic or structural elements of the virus, assembly of the components, or up-regulation of viral or host genes involved in the process.

General Techniques

Methods in molecular genetics and genetic engineering are described generally in the current editions of Molecular Cloning: A Laboratory Manual, (Sambrook et al.); Oligonucleotide Synthesis (M. J. Gait, ed.); Animal Cell Culture (R. I. Freshney, ed.); Gene Transfer Vectors for Mammalian Cells (Miller & Calos, eds.); Current Protocols in Molecular Biology and Short Protocols in Molecular Biology, 3rd Edition (F. M. Ausubel et al., eds.); and Recombinant DNA Methodology (R. Wu ed., Academic Press).

For general principles in vector construction, the reader is referred to Viral Vectors: Basic Science and Gene Therapy (Arrequi & Garcia-Carranca eds., Eaton Pub. Co., 2000). Reagents, cloning vectors, and kits for genetic manipulation referred to in this disclosure are available from commercial vendors such as BioRad, Stratagene, Invitrogen, and ClonTech.

For a description of the molecular biology of cancer, the reader is referred to Principles of Molecular Oncology (M. H. Bronchud et al. eds., Humana Press, 2000); The Biological Basis of Cancer (R. G. McKinnel et al. eds., Cambridge University Press, 1998); and Molecular Genetics of Cancer (J. K. Cowell ed., Bios Scientific Publishers, 1999).

General techniques for the development, testing, and administration of biomolecular chemotherapeutics are provided in Gene Therapy of Cancer, Adv. Exp. Med. Biol. vol. 451 (P. Walden ed., Plenum Publishing Corp., 1998); Cancer Gene Therapy, Adv. Exp. Med. Biol. vol. 465(N. A. Habib ed., Kluwer Academic Pub, 2000); and Gene Therapy of Cancer: Methods and Protocols, Meth. Mol. Med. vol. 35 (W. Walther & U. Stein eds., Humana Press, 2000).

Control Elements for Expression in the Target Cell Type

Exemplary vectors of this invention have a recombinant genome in which genes contained within the vector are placed under control of two different heterologous control elements—a telomerase-associated control element, and a second control element that is tissue or tumor specific.

Telomerase-Associated Control Element

Expression of at least one gene in the vector is under control of an transcriptional control element taken from a gene associated with telomerase activity. Suitable for this purpose are control elements of vertebrate genes that encode various components of the telomerase enzyme, such as the telomerase RNA component (U.S. Pat. No. 08/998,443). Also included are proteins that are believed to participate in telomerase regulation or telomere extension, or that are co-expressed with telomerase activity in cancer cells. Such proteins include Tankyrase I (WO 99/64696); Tankyrase II (WO 00/61813); TPC2 and TPC3 (U.S. Pat. No. 5,858,777).

Exemplary is a transcription control element for telomerase reverse transcriptase (TERT), the protein catalytic subunit of the telomerase enzyme. Sequence of the human TERT gene (including upstream promoter sequence) is provided below. International Patent Publication WO 00/46355 (Morin et al., Geron Corporation) describes and illustrates the construction and use of oncolytic virus (for example, adenovirus or HSV) that conditionally replicate under control of the human TERT promoter. The reader is also referred to U.K. Patent GB 2321642 B (Geron Corporation and U. Colorado), International Patent Publication WO 99/33998 (Bayer Aktiengesellschaft), and Horikawa et al. (Cancer Res., 59:826, 1999).

A lambda phage clone designated λGΦ5, containing ˜13,500 bases upstream from the hTERT encoding sequence, is available from the ATCC under Accession No. 98505. Suitable TERT promoter may comprise a sequence of 50, 100, or 200 consecutive nucleotides that is 80%, 90%, or 100% identical (or can hybridize under stringent conditions) to a sequence contained in SEQ. ID NO: 1. Short-length control elements are desirable because they fit within the packaging limit of the vector. For example, the TERT transcriptional control element may contain a minimal amount of the sequence from position −239 or −117 to position −36 or +1 relative to the translation initiation site (position 13545) of SEQ. ID NO: 1, or hybridize to such sequences under stringent conditions, and has the characteristic of preferentially promoting transcription in cells expressing TERT. Example 1 illustrates the testing and use of short TERT promoter sequences in vector expression systems.

A particular aspect of this invention relates to modification of the TERT promoter in such a way as to improve its activity or specificity in any context for any desirable purpose. Binding sites for transcriptional regulators are present in the sequence (see Table 1 of WO 00/46355 and U.S. Ser. No. 09/615,039, which is hereby incorporated herein by reference). Included are the ERE, MZF-2, WT1, SP1 sites and an E-box. Positive regulators believed to interact with the TERT promoter include Sp1, MYC/MAX and estrogen. Negative regulators include WT1, MZF-2, MAD1/MAX, HDAC and p53.

By deleting or modifying these recognition sites so as to affect binding of the transcriptional regulators, activity and specificity of the TERT promoter may be adjusted as needed. For example, interfering with certain negative regulators (especially those selectively expressed in cancer cells) may further increase transcription frequency, thereby improving activity. Interfering with certain positive regulators (especially those not expressed in cancer cells) may decrease transcription frequency in normal cell types, thereby improving specificity. Example 4 provides an illustration in which the E2F-1 binding site which down-regulates the TERT promoter is mutated to improve activity in cancer cells.

Promoter sequences not contained in λGΦ5 but homologous and capable of promoting preferential expression in cancer cells can be used with similar effect. Suitable are transcription control elements of other vertebrate TERT genes, and variants and homologs of vertebrate TERT genes that promote transcription in cancer cells expressing TERT. The mouse promoter and encoding TERT sequence is provided in WO 00/46355 (Geron Corporation) and at GenBank Accession No. AF121949.

Other Control Elements That are Tissue or Tumor Specific

In addition to a gene controlled by a TERT regulatory element, the vector of this invention can also contain a second gene controlled by a second heterologous transcriptional control element that is either tissue or tumor specific.

Tissue specific transcription control elements cause transcription to occur preferentially in cells of a particular tissue type: for example, tissues of a particular organ (such as liver, CNS, prostate, cardiac); or tissues of related type or derivation (such as mesenchymal cells or cells of hematopoietic origin). This means that the level of transcription with be at least 5-fold higher (and more typically 25- or 100-fold higher) in cells of the target tissue, in comparison with unrelated tissues from a mammal of the same species.

Examples of tissue-specific transcription control elements are promoters and enhancers that control transcription of albumin (liver-specific), α-fetoprotein (AFP, liver-specific), prostate-specific antigen (PSA, prostate-specific), mitochondrial creatine kinase (MCK, muscle-specific), myelin basic protein (MBP, oligodendrocyte-specific), glial fibrillary acidic protein (GFAP, glial cell specific), and neuron-specific enolase (NSE, neuron-specific). See U.S. Pat. No. 5,871,726 (Calydon), WO 98/39466 (Calydon), U.S. Pat. No. 5,998,205 (Genetic Therapy Inc.), and WO 99/25860 (Genetic Therapy Inc.). A subset of these tissue-specific elements cause transcription to occur preferentially in relatively undifferentiated, dedifferentiated, or transformed cells of the tissue type in question. One example is PSA, which is normally expressed exclusively in the prostate, but only at low levels, and becomes elevated in hyperplasias such as BPH and prostate cancer, and in metastases thereof.

Tumor specific transcription control elements are another category. Promoters that fall in this category cause transcription to occur in malignant cells of a plurality of tissue types at a higher level (at least 5-fold higher, more typically 25- or 100-fold higher) than in non-malignant cells of the same tissue type. Examples are promoters that control telomerase reverse transcriptase (TERT), carcinoembryonic antigen (CEA), hypoxia-responsive element (HRE), autocrine motility factor receptor (Grp78), L-plastin, and hexokinase II.

For purposes of providing dual specificity control in combination with the TERT promoter (described in the last section), of particular interest are transcriptional control elements for other components of the telomerase holoenzyme, or which are coexpressed with telomerase with a similar specificity for cancer cells. Exemplary is the promoter for the telomerase RNA component, which is also preferentially expressed in cancer cells. The promoter for human telomerase RNA component (hTR) is contained in the upstream sequence of SEQ. ID NO: 2. Telomerase RNA component sequence for other mammals is provided in Chen, Greider et al., Cell 100:503, 2000. Coexpressed with telomerase are the proteins designated TPC2 and TPC3 (U.S. Pat. No. 6,300,110). The upstream sequence for TPC2 is given in GenBank Accession No. AC004601.

Additional promoters suitable for use in this invention can be taken from other genes that are preferentially expressed in particular tissues or in particular tumor cells. Such genes can be identified, for example, by differential display and comparative genomic hybridization: see U.S. Pat. Nos. 5,759,776 and 5,776,683. Alternatively, microarray analysis can be performed by comparing mRNA preparations from cancer cells and a matched non-malignant control. Preferably, the level of expression of the effector gene will be at least 5-fold or even 25-fold higher in the target cell type relative to other cell types. Having identified transcriptional control elements of interest, specificity can be tested in a reporter construct where the control element is used to control transcription of a reporter gene (Example 1).

Construction of Dual-Controlled Vectors

The vectors of this invention comprise transcription control elements operatively linked to genes that have the effect of causing lysis, apoptosis, or attrition of the target cell. This effect can be achieved in viral vectors by linking the control elements to viral replication genes. When the vector is transduced into the appropriate target, replication is activated, and replication of the virus leads to lysis of the cell. Alternatively or in addition, the transcription control elements can be linked to particular genes that are directly toxic to the cell or otherwise affects the ability of the cell to survive.

Oncolytic Adenovirus Vectors

Certain vectors of this invention are replication competent adenovirus vectors, in which at least one gene essential for replication or assembly of the virus is placed under either a telomerase replication control element, or a tissue or tumor specific replication control element.

Adenoviruses are nonenveloped, regular icosahedrons. Early in viral replication, the E1a gene is translated, producing a pleomorphic protein that is a transactivator for other early genes (E1b, E2, E3, and E4). The E1b gene product acts on the host nucleus to alter function of the host cell such that processing and transport are shifted to the late genes, which cause packaging of the virus into its capsid and release from the cell. Adenovirus DNA includes inverted terminal repeat sequences (ITRs) of ˜100-150 base pairs, which enables single strands of viral DNA to circularize by base pairing of the termini. There is also a packaging signal of a few hundred base pairs, and both the ITRs and the packaging signal are required for packaging the replicated genome into an adenovirus particle.

The specificity of a telomerase transcription control element and a tumor or tissue specific transcription control element can be coupled in the same vector by operatively linking each to a different viral replication gene. Particularly suitable are the genes E1a, E1b, E2, E4, and the structural proteins controlled in the wild-type virus by the major late promoter—in various combinations as appropriate. For example, where the tissue specific control element causes transcription to occur preferentially in a particular cell type but not necessarily in tumor cells, then it may be preferable to link the E1a region to the telomerase control element. This way, the E1a region, which is thought to have undesirable effects for the host cell, will not be expressed in most normal tissue. The tissue specific control element can then be linked to another gene essential for replication or assembly of the virus, of which E4 and E1b are of particular interest. Full assembly of the virus (and consequent lysis of the cell) will only occur in telomerase-expressing cells of the target tissue type.

A further mechanism to prevent E1a expression outside the target cell is to replace it in the vector with a heterologous gene capable of providing the same function required for viral assembly. This invention includes adenovirus vectors in which any of the genes required for viral replication or assembly is replaced with a heterologous gene. Of particular interest for functional replacement are the adenovirus early genes, especially E1a, E1b, E2 and E4. E1a can be functionally replaced by a select group of transactivators also capable of promoting transcription of E1b, E2, and E4, that typically also modulate endogenous gene expression in the host cell. E2 can be replaced by an encoding region for one or more proteins that mimic the function of the E2 gene products: a single-stranded DNA binding protein, a DNA polymerase, and a terminal protein.

Candidate transactivators to replace E1a include viral transactivator genes from other viruses, such as members of the herpes simplex virus family, and SV40. Of particular interest are the immediate early genes from cytomegaloviruses (CMV) that are cytopathic for humans or other vertebrates—including the genes known as IE1 and IE2 (SEQ. ID NO: 4). Immediate early genes function to regulate viral and cellular gene expression during the course of CMV replication. The major IE region of the CMV genome is believed to activate viral genes and represses genes of the host cell. The molecular biology of CMV is reviewed by Emery et al. (Int. J. Exp. Pathol. 71:905, 1990) . Function and regulation of CMV IE genes are reviewed by Stenberg (Intervirology 39:343; 1996); Meier et al. (Intervirology 39:331; 1996); Spector (Intervirology 39:361; 1996); Spector et al. (Virology 151:329; 1986); and Tevethia et al. (Virology 161:276, 1987).

Other candidate transactivators include transactivator genes from higher eukaryotes, especially humans. Of particular interest is the family of Y box transactivators, including YB-1 (SEQ. ID NOS: 3 & 4). The specificity of the Y box transactivators is reviewed by R. Mantovani (Nucl. Acids Res. 26:1135, 1998 and Swamynathan et al. (FASEB J. 12:515, 1998). Didier et al. (Proc. Natl. Acad. Sci. USA 85:7433, 1988) investigated the cis-acting elements that regulate HLA Class II gene expression through the Y box (containing an inverted CCAAT box). By probing a phage λgt11 library with double-stranded oligonucleotides, they isolated cDNA for YB-1. It encodes a 35,414 kDa protein that has an absolute requirement for the CCAAT box and relative specificity for the Y box. There is an inverse correlation of YB-1 and HLA-DR β chain expression. YB-1 interacts with proliferating cell nuclear antigen (Ise et al., Cancer Res. 49:342, 1999), and may translocate to the nucleus by a protein kinase C mediated signal transduction pathway (FEBS Lett. 417, 390, 1997). YB-1 expression can be modulated by antisense compounds (U.S. Pat. No. 6,140,126).

Rather than having both the telomerase transcription control element and the tissue or tumor specific element drive viral replication genes, the specificity of the two elements can be coupled by having one drive transcription of a viral replication gene, and the other drive transcription of a heterologous gene that is directly toxic to the target cell or otherwise affects the ability of the cell to survive. Exemplary effector genes are described below.

The manipulation and molecular biology of adenovirus vectors is generally described in Adenovirus Methods and Protocols, Methods in Molecular Medicine Vol. 21, (W. S. M. Wold ed., Humana Press, 1998), and the current edition of Field's Virology (Lippincott Williams & Wilkins, 1996). Other publications of interest include Danthinne et al., Gene Ther. 7:1707, 2000, Bilbao et al., Adv. Exp. Med. Biol. 451:365, 1998, and U.S. Pat. Nos. 5,631,236 (Baylor College of Medicine), 5,670,488 (Genzyme), 5,698,443 (Calydon), 5,712,136 (GenVec), 5,880,102 (Duke University), 5,994,128 (IntroGene), 6,040,174 (Transgene), and 6,096,718 (Gene Targeting Corp).

The adenovirus vectors of this invention can be built using adenovirus capable of infecting any mammalian cell, including but not limited to human adenovirus—modeled on adenovirus type 2 or type 5, and readily adapted to other adenovirus types by substituting the genetic homologs wherever necessary or desired.

Oncolytic Herpesvirus Vectors

Certain vectors of this invention are replication competent herpesvirus vectors, in which at least one gene essential for replication or assembly of the virus is placed under either a telomerase replication control element, or a tissue or tumor specific replication control element.

Herpesviruses have an electron dense core harboring the double stranded DNA viral genome; a protein capsid surrounding the virus core comprised of 162 capsomeres; an amorphous layer surrounding the capsid termed the tegument; and a lipid envelope containing spikes thought to be viral glycoproteins. They encode many enzymes involved in nucleic acid metabolism, they replicate and assemble in the nucleus, and the host cell is lysed as an outcome of virus infection. Five HSV genes: a4-ICP4, a0-ICP0, a27-ICP27/UL54, a22-ICP22/US1, and a47-ICP47/US12 are expressed and function the earliest stages of the productive infection cycle. This stage of infection is termed the “immediate-early” or “a” phase of gene expression. Activation of the host cell transcriptional machinery by the action of a gene products, results in the expression of the “early” or “β” genes. Seven of these are necessary and sufficient for viral DNA replication under all conditions: DNA polymerase (UL30), DNA binding proteins (UL42 and UL29 or ICP8), ORI binding protein (UL9), and the helicase/primase complex (UL5, 8, and 52).

The strategy for assembling an oncolytic herpesvirus vector of this invention parallels the strategy outlined above for assembling adenovirus vectors. The specificity of a telomerase transcription control element and a tumor or tissue specific transcription control element can be coupled in the same vector by operatively linking each to a herpes replication gene: for example, ICP0 and ICP4, or any other combination of genes required for replication or assembly of the vector.

Rather than having both the telomerase transcription control element and the tissue or tumor specific element drive viral replication genes, the specificity of the two elements can be coupled by having one drive transcription of a viral replication gene, and the other drive transcription of a heterologous gene that is directly toxic to the target cell or otherwise affects the ability of the cell to survive. Exemplary effector genes are described in the section that follows.

The molecular biology of herpesvirus vectors is generally described in The organization of the herpes simplex virus genomes (B. Roizman, Annu. Rev. Genet. 13:25, 1979); Herpes Simplex Virus Protocols (Brown et al. eds., Humana Press 1998) and Herpesvirus Transcription and its Regulation (E. K. Wagner ed., CRC Press 1991). For general descriptions of the construction and testing of replication competent virus, see also U.S. Pat. Nos. 5,585,096; 5,728,379; and 6,139,834 (Martuza et al.).

Of course, this invention also contemplates replication-competent oncolytic vectors comprising components of viruses other than those of the adenovirus or herpesvirus family. Under certain circumstances, vectors built from a papovavirus, papillomavirus, or hepaDNA virus may also be effective. The strategy for assembling a vector of this invention based on other viruses follows mutatis mutandis the strategy outlined above for assembling adenovirus and herpesvirus vectors, using replication genes appropriate for the virus type being used.

Other Mechanisms for Vector-Induced Cell Killing

Alternatively or in addition, the specific transcription control elements can be linked to particular genes that are directly toxic to the cell or otherwise affects the ability of the cell to survive.

One type of effector gene that can be used for this purpose is a gene that encodes a peptide toxin, such as ricin, diphtheria toxin, or a spider venom neurotoxin (Escoubas et al., Biochimie 82:893, 2000). Other suitable effectors encode polypeptides having activity that is not directly toxic to a cell, but renders the cell sensitive to an otherwise nontoxic compound. Exemplary is thymidine kinase, which converts the anti-herpetic agent ganciclovir to a toxic product that interferes with DNA replication in proliferating cells (U.S. Pat. No. 5,631,236 and EP 657541 A1). Other illustrations (reviewed by Aghi et al., J. Gene Med. 2:148, 2000) are cytosine deaminase (which activates agents such as 5-fluorocytosine) and purine nucleoside phosphorylase.

An alternative type of effector gene encodes a gene product that induces or mediates apoptosis. Exemplary are Caspase-1, Caspase-3, Caspase-8, and Bax.

An alternative type of effector gene causes a cell in which it is expressed to become more susceptible to the effects of the immune or inflammatory response. For example, the gene for α(1,3)galactosyltransferase (Henion et al., Glycobiology 4:193, 1994) causes expression of the Galα(1,3)Gal xenoantigen on human tissues, which then becomes a target for complement lysis mediated by circulating naturally occurring antibody. The A- and B-transferase enzymes can be used in subjects of opposite blood groups with similar effect (WO 02/42468).

A further type of effector gene is a gene that encodes a cytokine or other regulator of inflammation, immunity, cell growth, or angiogenesis. The mediator is secreted from the target cell, and triggers other cells in the milieu to take more evasive action against the tumor. Included are interferons, interleukins, tumor necrosis factors, and anti-angiogenesis agents.

A further alternative type of effector gene is a tumor suppressor gene. There are 94 known human tumor suppressor genes, of which 82 have corresponding UniGene entries and 55 match one or more CGAP sequences. The genes p53 and RB are exemplary. For other examples, the reader is referred to The Oncogene and Tumour Suppressor Gene Facts Book (R. Hesketh, 2^{nd} edition, Academic Press, 1997).

Using effector genes of this nature, specificity of the telomerase transcription control element and the tissue or tumor specific control element can be coupled in several different fashions. One system is to have a control element drive a viral replication gene, and the other element drive the effector gene. For example, a liver-specific adenovirus vector could have a TERT promoter controlling E1a expression, and an albumin promoter controlling expression of a caspase. Another system is to have both elements control functional expression of the same gene product. For example, the first control element could drive expression of a neurotoxin in a precursor form, and the second element could drive expression of an enzyme that converts it to the active form. Thus, the two control elements drive expression of a pair of gene products that are only lethal to the cell when expressed concurrently. Where vector replication is not required for lethality or to penetrate the target tissue, the vector need not be replication competent, and need not be based on a viral genome.

Effector Agents that are Specific for the Target Cell Type

Multiple specificity can also be achieved by combining one or more tissue or tumor specific promoters with an effector gene that specifically affects the viability of cancer cells.

Exemplary are encoding regions that produce a transcript that affects telomerase activity in the cell. The transcript can target mRNA encoding the telomerase protein component (TERT), preventing it from being translated and forming functional telomerase holoenzyme. Alternatively, the transcript can target the telomerase RNA component (hTR, SEQ. ID NO: 2), which can inhibit telomerase activity either by preventing hTR from associating with TERT, by preventing the holoenzyme from binding to telomeres, or preventing telomere extension once bound.

A polynucleotide effective in targeting hTR or TERT can be in the form of complementary sequences that duplex with the RNA with sufficient avidity and specificity to prevent activity. The sequences typically comprise at least 10, 20, 30, or 50 consecutive nucleotides that are complementary to the naturally occurring hTR or TERT sequence (WO 99/50279). The polynucleotide can also be in the form of a ribozyme that specifically cleaves the hTR or the mRNA for TERT (WO 99/50279). Also suitable are specific effector sequence is based on RNA interference (RNA;) technology (Sharp et al., Genes Dev. 13:139, 1999; Tavernarakis et al.; Nat. Genet. 24:180, 2000). The RNAi transcript comprises inverted repeats taken from the hTR or TERT mRNA sequence, separated by a short linker sequence. It forms a hairpin structure with a double-stranded region, inducing stable and inheritable RNAi effects, and causing the target RNA to be destroyed (WO 02/42445).

Exemplary tissue specific effector genes are a modified synthetic amoebapore helix 3 peptide, lethal for prostate cancer (Warren et al., Cancer Res. 61:6783, 2001); GnRH-Bik/Bax/Bak chimeric proteins, lethal for adenocarcinoma (Azar et al., Apoptosis 5:531, 2000), and overexpression of Fas ligand, lethal for neuroblastoma cells (Takamizawa et al., J. Pediatr. Surg. 35:375, 2000).

The use of these effectors provides a degree of target cell specificity beyond the specificity driven by the target cell specific control elements. One embodiment of this invention is a vector comprising a tumor or tissue specific effector sequence, and at least one tumor or tissue specific transcription control element that is heterologous to the rest of the vector construct. The transcription control element can drive transcription of the specific effector, providing dual specificity in one promoter effector combination. In this case, the vector need not be replication competent, and can be based on a viral backbone or synthetic construct, such as a lipid DNA complex (e.g., U.S. Pat. No. 6,410,328). Alternatively, the tumor or tissue specific effector sequence can be under the control of a constitutive promoter (such as the promoter for CMV, SV40, β-actin, ubiquitin, EF1a, or PGK). This can be combined in the vector with a gene required for replication or assembly of the vector, driven by a tumor specific transcription control element exemplified by a promoter for TERT or hTR.

Of course, one or more tissue or tumor specific transcriptional control elements can be combined with one or more tissue or tumor specific effector genes in any effective combination to provide synergistic specificity to any desired extent.

Formulation and Testing of Tumor-Killing Vectors

Whether the transcription control elements and effector genes used in a particular vector construct are suitable for the intended objective can be determined by standard screening methods.

Initial screening can be performed most conveniently using cultured target cells and control cells in vitro. Malignant and non-malignant cell lines of the target tissue type and other tissue types are transduced with the test constructs over a range of particle densities, and lysis or apoptosis of the cells is determined during the subsequent culture. Example 2 illustrates the evaluation of oncolytic virus by testing the effect on a panel of cancer cell lines, and comparing with the effect on other cell lines. Example 3 illustrates quantitation of specificity, by determining the number of pfu produced per cell, standardizing using a positive vector control, and comparing between different cell lines.

Further validation of a virus of this invention for the treatment of a particular condition can be tested in a suitable animal model. For example, efficacy for treating cancer can be determined using mice injected with a representative human cancer cell line, such as a glioma or osteosarcoma. After solid tumors have developed of a sizeable diameter, the mice are injected intravenously or intratumorally with the chimeric vector, for example, in a dose range of 10⁶ to 10⁹ pfu, and then monitored for reduced tumor growth rate and increased survival (Example 5).

Dosage and formulation of medicaments intended for human therapy are based on the animal model experiments. For general guidance on formulation and testing of medicament formulations for human administration, the reader is referred to Biopharmaceutical Drug Design and Development (S. Wu-Pong et al. eds, Humana Press 1999); Biopharmaceuticals: Biochemistry and Biotechnology (G. Walsh, John Wiley & Sons, 1998); and the most current edition of Remington: The Science and Practice of Pharmacy (A. Gennaro, Lippincott, Williams & Wilkins).

The compositions of this invention can be used to treat any condition (either benign or malignant) that is associated with overexpression of a telomerase component such as TERT. Benefit may ensue from the agent alone, or in combination with other accepted therapeutic regimens, such as chemotherapy or radiation therapy. The pharmaceutical compositions of this invention may be packaged in a container with written instructions for use of the cells in human therapy, and the treatment of cancer.

### The examples that follow are provided by way of further illustration, and are not meant to limit the claimed invention.

### EXAMPLES

### Example 1

### Preparation of Vectors Controlling Transcription in Cells Expressing Telomerase Reverse Transcriptase

The lambda clone designated λGΦ5 containing the hTERT promoter is deposited with the American Type Culture Collection (ATCC), 10801 University Blvd., Manassas, Va. 20110 U.S.A., under Accession No. 98505. λGΦ5 contains a 15.3 kbp insert including approximately 13,500 bases upstream from the hTERT coding sequence. Further details regarding the sequencing and testing of the hTERT gene are provided in WO 00/46355.

A Not1 fragment containing the hTERT promoter sequences was subcloned into the Not1 site of pUC derived plasmid, which was designated pGRN142. A subclone (plasmid pGRN140) containing a 9 kb NcoI fragment (with hTERT gene sequence and about 4 to 5 kb of lambda vector sequence) was partially sequenced to determine the orientation of the insert. pGRN140 was digested using Sail to remove lambda vector sequences, the resulting plasmid (with removed lambda sequences) designated pGRN144. The pGRN144 insert was then sequenced.

SEQ. ID NO: 1 is a listing of the sequence data obtained. Nucleotides 1-43 and 15376-15418 are plasmid sequence. Thus, the genomic insert begins at residue 44 and ends at residue 15375. The beginning of the cloned cDNA fragment corresponds to residue 13490. There are Alu sequence elements located ˜1700 base pairs upstream. The sequence of the hTERT insert of pGRN142 can now be obtained from GenBank (http://www.ncbi.nlm.nih.gov/) under Accession PGRN142.INS AF121948. Numbering of hTERT residues for plasmids in the following description begins from the translation initiation codon, according to standard practice in the field. The hTERT ATG codon (the translation initiation site) begins at residue 13545 of SEQ. ID NO: 1. Thus, position −1, the first upstream residue, corresponds to nucleotide 13544 in SEQ. ID NO: 1.

Expression studies were conducted with reporter constructs comprising various hTERT upstream and intron sequences. A BgIII-Eco47III fragment from pGRN144 (described above) was digested and cloned into the BgIII-NruI site of pSEAP2Basic (ClonTech, San Diego, Calif.) to produce plasmid designated pGRN148. A second reporter-promoter, plasmid pGRN150 was made by inserting the BgIII-FspI fragment from pGRN144 into the BgIII-NruI sites of pSEAP2. Plasmid pGRN173 was constructed by using the EcoRV-StuI (from+445 to −2482) fragment from pGRN144. This makes a promoter reporter plasmid that contains the promoter region of hTERT from approximately 2.5 kb upstream from the start of the hTERT open reading frame to just after the first intron within the coding region, with the initiating Met codon of the hTERT open reading frame changed to Leu. Plasmid pGRN175 was made by APA1(Klenow blunt)-SRF1 digestion and religation of pGRN150 to delete most of the Genomic sequence upstream of hTERT. This makes a promoter/reporter plasmid that uses 204 nucleotides of hTERT upstream sequences (from position −36 to −117). Plasmid pGRN176 was made by PML1-SRF1 religation of pGRN150 to delete most of the hTERT upstream sequences. This makes a promoter/reporter plasmid that uses 204 nucleotides of hTERT upstream sequences (from position −36 to −239).

Levels of secreted placental alkaline phosphatase (SEAP) activity were detected using the chemiluminescent substrate CSPDTM (ClonTech). SEAP activity detected in the culture medium was found to be directly proportional to changes in intracellular concentrations of SEAP mRNA. The pGRN148 and pGRN150 plasmids (hTERT promoter-reporter) and the pSEAP2 plasmid (positive control, containing the SV40 early promoter and enhancer) were transfected into test cell lines. pGRN148 and pGRN150 constructs drove SEAP expression as efficiently as the pSEAP2 in immortal (tumor-derived) cell lines. Only the pSEAP2 control gave detectable activity in mortal cells.

The ability of the hTERT promoter to specifically drive the expression of the thymidine kinase (tk) gene in tumor cells was tested using a variety of constructs: One construct, designated pGRN266, contains an EcoRI-FseI PCR fragment with the tk gene cloned into the EcoRI-FseI sites of pGRN263. pGRN263, containing approximately 2.5 kb of hTERT promoter sequence, is similar to pGRN150, but contains a neomycin gene as selection marker. pGRN267 contains an EcoRI-FseI PCR fragment with the tk gene cloned into the EcoRl-FseI sites of pGRN264. pGRN264, containing approximately 210 bp of hTERT promoter sequence, is similar to pGRN176, but contains a neomycin gene as selection marker. pGRN268 contains an EcoRI-XbaI PCR fragment with the tk gene cloned into the EcoRI-XbaI (unmethylated) sites of pGRN265. pGRN265, containing approximately 90 bp of hTERT promoter sequence, is similar to pGRN175, but contains a neomycin gene as selection marker.

These hTERT promoter/tk constructs, pGRN266, pGRN267 and pGRN268, were re-introduced into mammalian cells and tkl+ stable clones (and/or mass populations) were selected. Ganciclovir treatment in vitro of the tkl+ cells resulted in selective destruction of all tumor lines tested, including 143B, 293, HT1080, Bxpc-3′, DAOY and NIH3T3. Ganciclovir treatment had no effect on normal BJ cells.

### Example 2

### Killing Cancer Cells using Vectors Controlled by the TERT Promoter

A replication-conditional adenovirus was constructed by placing a gene involved in viral replication under control of the hTERT promoter, which should activate transcription in telomerase-expressing cancer cells. The viral construct comprised the Inverted Terminal Repeat (ITR) from adenovirus Ad2; followed by the hTERT medium-length promoter (phTERT176) operably linked to the adenovirus E1a region; followed by the rest of the adenovirus deleted for the E3 region (ΔE3). As a positive control, a similar construct was made in which E1a was placed under control of the CMV promoter, which should activate transcription in any cell.

Reagents were obtained as follows. pBR322, restriction enzymes: NEB, Beverly, Mass. Adenovirus Type 2 (Ad2), tissue culture reagents: Gibco/BRL, Grand Island, N.Y. Profection Mammalian Transfection Systems™: Promega, Madison, Wis. Tumor and Normal Cell lines: ATCC, Manassas, Va., except BJ line, which was obtained from J. Smith, U. of Texas Southwestern Medical Center.

Briefly, a pBR322-based plasmid was constructed which contains the Adenovirus Type 2 genome with deletions from 356-548nt (E1a promoter region) and 27971-30937nt (E3). A multiple cloning region was inserted at the point of deletion of the E1a promoter, and hTERT promoter (−239 to −36nt) or CMV promoter (−524 to −9nt) was subsequently cloned. Numbering of the CMV sequence is in accordance with Akrigg et al., Virus Res. 2:107, 1985. Numbering of the Ad2 sequence is in accordance with “DNA Tumor Viruses: Molecular Biology of Tumor Viruses”, J. Tooze ed., Cold Spring Harbor Laboratory, N.Y.

These plasmid DNAs were digested with SnaBI to liberate ITRs, then phenol-chloroform extracted, precipitated and transfected into 293A cells for propagation of the virus. Several rounds of plaque purifications were performed using A549 cells, and a final isolate was expanded on these same cells. Viruses were titered by plaque assay on 293A cells, and tested for the presence of 5′ wild type Ad sequences by PCR. DNA was isolated from viruses by HIRT extraction.

The vector comprises the Inverted Terminal Repeat (ITR) from the adenovirus (Ad2); followed by the hTERT medium-length promoter (phTERT176) operably linked to the adenovirus E1a region; followed by the rest of the adenovirus deleted for the E3 region (ΔE3). Modified constructs are also possible. One comprises an additional sequence in between the hTERT promoter and the E1a region. The HI sequence is an artificial intron engineered from adenovirus and immunoglobulin intron splice donor and acceptor sequences. It is thought that placing an intron in the hTERT promoter adenovirus replication gene cassette will promote processing and transport of heteronuclear RNA, thereby facilitating formation of the replicated viral particles.

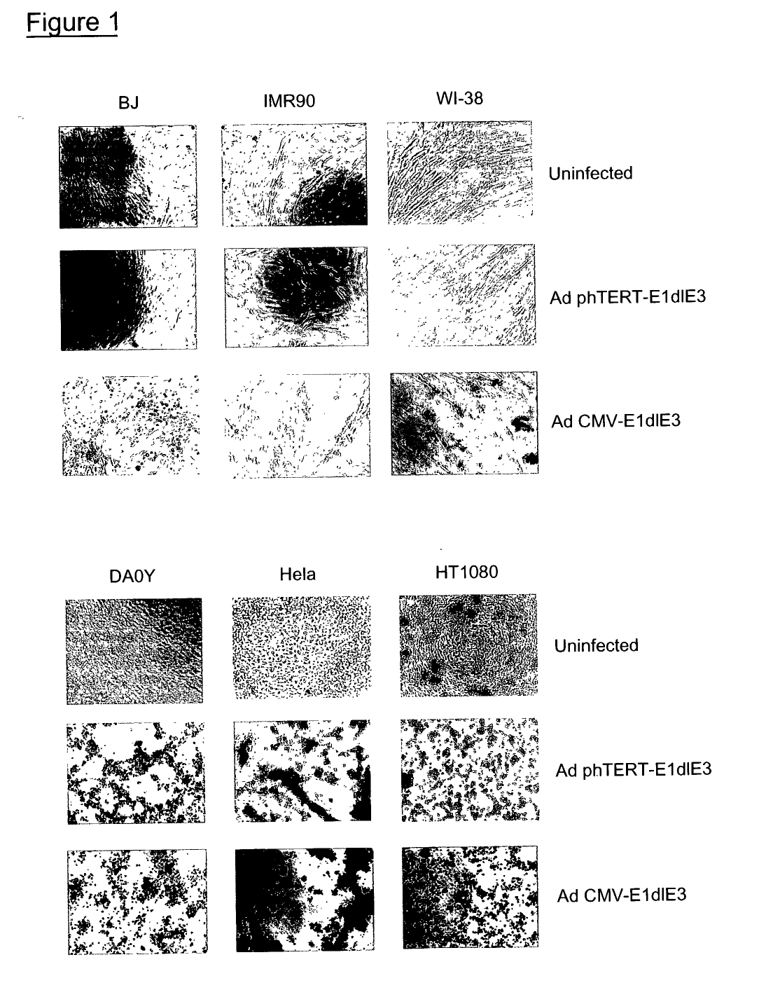

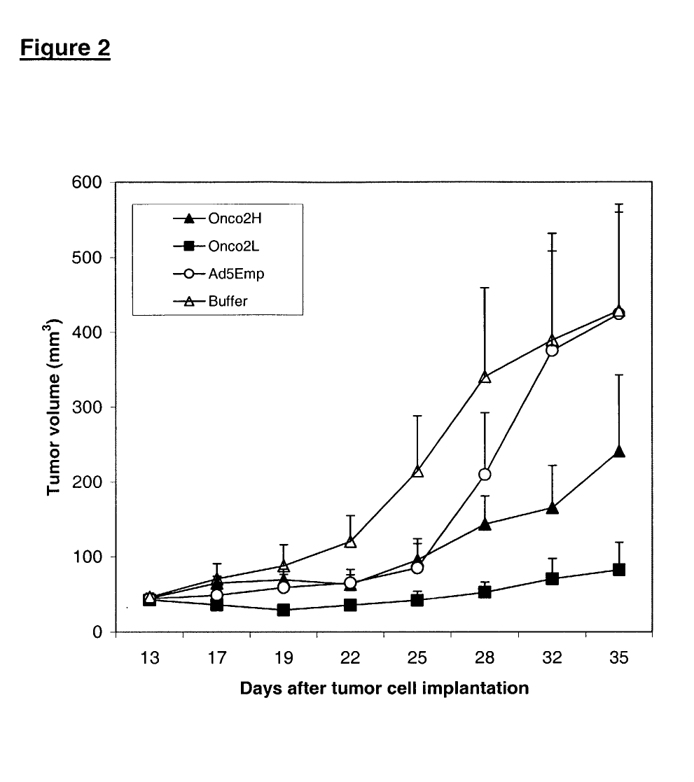

FIG. 1 shows the effect of these viruses on normal and cancer-derived cell lines. Each cell line was plated and infected at an MOI=20, ˜24 h post plating. The cells were then cultured over a period of 17-48 days, and fed every fourth day. The pictures shown in the Figure were taken 7 days after infection. The top row of each section shows the results of cells that were not virally infected (negative control). The middle row shows the results of cells infected with oncolytic adenovirus, in which replication gene E1a is operably linked to the hTERT promoter. The bottom row of each section shows the results of cells infected with adenovirus in which E1a is operably linked to the CMV promoter (positive control). Results are summarized in Table 1.

TABLE 1
Effect of Oncolytic Virus on Cancerous and Non-cancerous Cells

| | | | Uninfected | Lysis by | Lysis by |
|---|---|---|---|---|---|
| | | | cell | phTERT- | pCMV- |
| Cell Line | Origin | Culture Conditions | Lysis | E1ΔE3 | E1ΔE3 |
| BJ | foreskin fibroblast | 90% DMEM/M199 + | NO | NO | YES |
| | | 10% FBS | | | |
| IMR | lung fibroblast | 90% DMEM/M199 + | NO | NO | YES |
| | | 10% FBS | | | |
| WI-38 | lung fibroblast | 90% DMEM/M199 + | NO | NO | YES |
| | | 10% FBS + | | | |
| | | 5 μg/mL gentamycin | | | |
| A549 | lung carcinoma | 90% RPMI + | NO | YES | YES |
| | | 10% FBS | | | |
| AsPC-1 | adenocarcinoma, | 90% RPMI + | NO | YES | YES |
| | pancreas | 10% FBS | | | |
| BxPC-3 | adenocarcinoma, | 90% EMEM + | NO | YES | YES |
| | pancreas | 10% FBS | | | |
| DAOY | medulloblastoma | 90% EMEM + | NO | YES | YES |
| | | 10% FBS | | | |
| HeLa: | cervical carcinoma | 90% EMEM + | NO | YES | YES |
| | | 10% FBS | | | |
| HT1080 | fibrosarcoma | 90% EMEM + | NO | YES | YES |
| | | 10% FBS | | | |

All cell lines tested were efficiently lysed by AdCMV-E1dlE3 by day 17 post-infection. All tumor lines were lysed by AdphTERT-E1dlE3 in a similar, but slightly delayed period, while normal lines showed no signs of cytopathic effect and remained healthy out to 6 weeks post-infection.

The results demonstrate that an oncolytic virus can be constructed by placing a genetic element essential for replication of the virus under control of an hTERT promoter. Replication and lysis occurs in cancer cells, but not in differentiated non-malignant cells.

### Example 3

### Replication-Conditional Adenovirus Regulated by Two Different Promoters

Two replication-competent adenovirus variants were constructed by transcriptional targeting of the E1a and E1b genes. Further details of this example are published by Yu et al., Cancer Res. 59:1493, 1999.

CV763 contained the enhancer domain (−5155 to −3387) and the proximal promoter −324 to +33) of the human kallikrein 2 gene (Yu et al., op. cit.) cloned at the E1a transcription start site to drive E1a expression. CV764 contains a copy of the PSE sequence (Rodriguez et al., Cancer Res. 57:2559, 1997) at the E1a transcription start site to drive E1a expression and a copy of hK2 transcription response element at the E1b transcription start site to drive E1b expression. CN702, an adenovirus that has a wild-type E1 region, was used as a wild-type control in this study. CN706 contains a copy of PSE at E1a transcription start site to drive E1a expression and showed selective cytotoxicity toward PSA expressing cells in vitro and in vivo. Virus structures were confirmed by PCR and Southern blotting and found to be genetically stable.

To determine whether the adenovirus variants described above replicate preferentially in prostate cancer cells, virus yield (in pfu/cell) assays were performed at 48 h post infection. Virus yield per cell was evaluated in the following cell types: human 293 cells, prostate tumor cell line (LNCaP), breast normal cell line (HBL-100), and ovarian tumor cell lines (OVCAR-3, SK-OV-3, and PA-1). 293 cells serve as a positive control because this cell line expresses Ad5 E1a and E1b proteins. LNCaP cells express both androgen receptor and PSA.

The viruses CN702, CN706, CV763, and CV764 equally produced 1×10⁴ pfu per cell in 293 cells. Slightly lower yields were found with the PSA(1) LNCaP cells in which CN702 produced 5×10³ pfu/cell, whereas CN706, CV763, and CV764 equally produced 1-2×10³ pfu/cell. However, the prostate specific ARCA variants CN706, CV763, and CV764 grew poorly on nonprostate cells. For example, CV763 and CN706 yielded 100-fold less virus/cell in HBL-100, OVCAR-3, SK-OV-3, and PA-1 cells than the wild-type E1 virus CN702. This indicates that the hK2 transcription response element engineered adenovirus preferentially replicates in prostate tumor cells.

CV764, a virus with PSE driving E1a and hK2 transcription response element driving E1b, is significantly replication restricted in nonprostate tumor cells. The virus yield (in pfu/cell) decreased by 5000-fold in HBL-100 cells, 8000-fold in PA-1 cells, and 10,000-fold in SK-OV-3 and OVCAR-3 cells when compared to CN702. Indeed, CV764 yielded<1 pfu/cell in all of the PSA(−) cells, a rate of replication that clearly cannot sustain an active self-sustaining virus replication.

To characterize the differential viral cytopathic effects in primary human cells, CPE assays were performed. Nonimmortalized hMVECs were chosen to test sensitivity to CV764 and wild-type adenovirus (CN702) infection. CN702 caused monolayer cytolysis of hMVEC monolayers at MOI as low as 0.01 within 10 days. In contrast, CV764 infected hMVEC monolayers did not show significant cytopathic effects at the same time points with MOI of 10, 1.0, 0.1, and 0.01. Cytolysis of hMVECs with CV764 equivalent to that seen with wild-type CN702 adenovirus was only evident at a MOI 1,000-10,000-fold greater than the MOI used with CN702.

Thus, CV764-mediated cytolysis is significantly attenuated relative to wild type adenovirus in primary normal hMVECs.

### Example 4

### Adjusting TERT Promoter Activity

E2F-1 is a transcription factor that in free form normally acts as a transcriptional activator. When complexed with retinoblastoma protein (pRB), it becomes a negative regulator (Dyson, Genes Dev. 12:2245, 1998). Many cancer cells have mutations in pRB, causing decreased complexing between pRB and E2F-1. Increased free E2F in pRB mutated cells has the paradoxical effect of down-regulating the TERT promoter through the E2F-1 binding site (Crowe et al., Nucl. Acids Res. 29:2789, 2001). It is hypothesized that the presence of functional E2F-1 binding sites in the TERT promoter means that cancer cells having higher free E2F-1 levels may actually repress promoter activity.

An improved human TERT promoter can be prepared with the object of increasing activity in cancer cells containing a pRB mutation. The E2F binding sites in the TERT promoter are altered to reduce binding activity by inserting point mutations. The modified promoter is then inserted into expression vectors to test the effect of the mutations on activity and specificity.

The modified TERT promoter is constructed as follows. Two PCR primers are synthesized, a forward primer containing the two mutated E2F sites, and a wild-type reverse primer. The forward primer consists of bases (5′) −280 to −110 (3′) (where+1 is defined as the initial A of the ATG start codon) of the genomic TERT promoter sequence (SEQ. ID NO: 1), where both E2F sites at −251 and −175 are modified from 5′-CGCGC-3′ to 5′-CGCct-3′. The reverse primer contains the genomic wild-type TERT sequence comprising bases (5′) −001 to −159 (3′). The two primers are combined in equimolar ratios and subjected to polymerase chain reaction using each other as templates to create the double-stranded modified promoter fragment.

Reporter constructs are made by cloning the amplified DNA into a pGLOW-TOPO™ expression vector from Invitrogen (Carlsbad Calif.), catalog # K4830-01, comprising a TOPO TA cloning® site for a promoter test construct upstream of a GFP reporter gene. Technical details of the cloning procedure are provided in the TOPO Reporter Kits Manual Version J, 011002, 25-0235. As a control, an unmodified TERT promoter construct is made the same way with the exception that the two E2F sites are left as unmodified wild-type sequence.

The promoter-reporter constructs are tested for activity and specificity using a suitable panel of cell lines. Total cellular RNA is isolated at different time-points post-infection and relative RNA expression levels are determined by quantitative RT-PCR, using TaqMan™ primers and probes to the GFP reporter gene by standard methods. It is expected that the E2F-modified TERT promoter constructs will have increased transcriptional activity in pRB−, TERT+ cancer cell lines relative to the unmodified TERT promoter constructs. The modified and un-modified constructs should have equivalent levels of expression when transfected into cell lines that are pRB+, TERT+. The transcriptional activity of both TERT promoter constructs should be much lower in normal (pRB+, TERT−) cell lines.

Modified promoter constructs having the desired activity and specificity are then used to construct and test a conditional replicative oncolytic adenovirus, as in Example 2.

### Example 5

### Reducing Tumor Growth In Vivo Using a TERT-Driven Oncovirus

This experiment illustrates efficacy testing of a replication-conditional cytolytic virus in an animal model. The virus used was an oncolytic adenovirus in which the E1a gene is placed under control of the hTERT promoter (Example 2). The 143B cell line is a human osteosarcoma, and was obtained from the ATCC.

Six to eight week old female BALB/c nude mice were injected subcutaneously in the flank with 2×10⁵ 143B cells with a Matrigel® support (Becton Dickinson). Tumors of ˜50 mm³ formed at the injection site by the 10^{th} day. The tumors were directly injected with the oncolytic virus in a volume of ˜50 μL daily from day 11 to day 15. Tumor size was monitored thereafter, and calculated assuming the shape of an ellipsoid body (L×W×H÷2).

FIG. 2 shows growth of tumors in these animals as a function of days after engrafting the 143B cells. Onco2H=oncolytic virus at a dose of 2.5×10⁸ pfu per mouse. Onco2L=oncolytic virus at a dose of 1×10⁸ pfu per mouse. Ad5Emp=adenovirus vector lacking E1a and E3 genes. Buffer=buffer alone (negative control). n≈10 mice in each group.

The combined results of these experiments demonstrate that an adenovirus construct that is replication-conditional under control of the hTERT promoter specifically kills cancer cells and slows the rate of tumor growth by about 2-fold to over 5-fold, depending on dose.

TABLE 2
Sequences listed in this Disclosure

| SEQ. ID NO: | Designation | Reference |
|---|---|---|
| 1 | Lambda clone designated λGφ5 | GenBank Accession AF121948. |
| | (ATCC Accession No. 98505) | International Patent Publication |
| | Human TERT encoding region & | WO 00/46355. |
| | upstream sequence. | |
| | Contains human Telomerase Reverse | |
| | Transcriptase (hTERT) genomic insert | |
| | (residues 44-15375). The ATG | |
| | translation initiation site begins at residue | |
| | 13545. | |
| 2 | The 2.5 kb HindIII-Sac* insert of plasmid | U.S. patents 6,054,575 and 6,320,039. |
| | pGRN33 | Feng et al., Science 269:1236, 1995; |
| | (ATCC Accession No. 75925) | GenBank Accession No. U86046. |
| | Human telomerase RNA component | |
| | encoding region & upstream sequence. | |
| | Contains the human telomerase RNA | |
| | component and upstream sequence. The | |
| | RNA component sequence begins at | |
| | base 1459. | |

The compositions and procedures provided in the description can be effectively modified by those skilled in the art without departing from the spirit of the invention embodied in the claims that follow.

What is claimed as the invention is:

1. A viral vector comprising a first gene controlled by a first heterologous transcriptional control element and a second gene controlled by a second heterologous transcriptional control element, wherein the first transcriptional control element causes the first gene to be preferentially expressed in cells expressing telomerase reverse transcriptase (TERT), and wherein transduction of the vector into a mammalian cell expressing TERT causes killing of the cell or its progeny.

2. The vector of the preceding claim, wherein the first gene is under control of a human TERT promoter.

3. The vector of claim 2, wherein the human TERT promoter has been adapted to delete or inactivate an E2F-1 binding site.

4. The vector of any preceding claim, wherein the second gene is under control of a heterologous transcriptional control element for a tissue or tumor specific gene other than TERT.

5. The vector of claim 4, wherein the second gene is under control of a transcriptional control element for a tissue specific gene selected from albumin, α-fetoprotein, prostate-specific antigen, mitochondrial creatine kinase, myelin basic protein, glial fibrillary acidic protein, and neuron-specific enolase.

6. The vector of claim 4, wherein the second gene is under control of a transcriptional control element for a telomerase RNA component (hTR).

7. The vector of any preceding claim, wherein the gene controlled by the first transcriptional control element is a gene required for replication or assembly of the vector.

8. The vector of any preceding claim, wherein the gene controlled by the second transcriptional control element is a gene required for replication or assembly of the vector.

9. The vector of any preceding claim, which is a replication-conditional adenovirus.

10. The vector of any preceding claim, wherein a gene controlled by the TERT transcriptional control element is contained within an adenovirus E1a, E1b, E2, or E4 region.

11. The vector of claims 1-5, which is a replication-conditional herpesvirus.

12. The vector of claims 1-8 or claim 11, wherein a gene controlled by the TERT transcriptional control element is a herpesvirus ICP0 or ICP4 gene.

13. The vector of any preceding claim, wherein a gene controlled by the tissue or tumor specific control element is contained within an adenovirus E1a, E1b, E2, or E4 region; or is a herpesvirus ICP0 or ICP4 gene.

14. The vector of any preceding claim, further comprising an encoding region whose expression is toxic to the cell, which renders the cell more susceptible to toxic effects of a drug, which encodes a cytokine, or which is a tumor supressor gene.

15. The vector of claim 14, wherein the encoding region encodes a thymidine kinase or a caspase.

16. The vector of any preceding claim, further comprising an encoding region whose expression causes a reduction of telomerase activity in the cell.

17. A vector comprising an encoding region whose expression causes a reduction of telomerase activity in the cell, under control of a promoter for TERT or telomerase RNA component.

18. The vector of claims 16-17, wherein the encoding region encodes a ribozyme, RNAi, or complementary polynucleotide that specifically inhibits translation of human TERT mRNA.

19. The vector of claims 16-17, wherein the encoding region encodes a ribozyme, RNAi, or complementary polynucleotide that specifically binds or degrades hTR, thereby inhibiting telomerase activity.

20. A conditionally replicative viral vector comprising a gene required for replication or assembly of the vector under control of a TERT promoter has been adapted to delete or inactivate an E2F-1 binding site.

21. A method for selecting a vector according to claims 1-19, comprising transducing a host cell with a vector comprising one gene under control of a transcriptional control element for a telomerase reverse transcriptase (TERT), and another gene under control of a heterologous transcriptional control element for a tissue or tumor specific gene other than TERT; and then determining any effect of the vector on the host cell.

22. A method for killing a cancer cell, comprising contacting the cell with the vector of claims 1-19.

23. A method of treating a subject for a condition associated with increased expression of telomerase reverse transcriptase in affected cells, comprising administering to the subject an effective amount of the vector according to claims 1-19.

24. Use of a vector of any of claims 1-19 in the preparation of a medicament for treatment of a condition associated with increased expression of telomerase reverse transcriptase.

25. The method or use of claim 23 or 24, wherein the condition is cancer.

26. The method or use of claims 22 or 25, wherein the cancer is selected from liver cancer, prostate cancer, muscle cancer, neural cell cancer, lung cancer, pancreatic cancer, medulloblastoma, cervical carcinoma, fibrosarcoma, and osteosarcoma.


SEQUENCE LISTING (2 sequences)

SEQ ID NO: 1 - DNA, 15418 bp, Homo sapiens
gcggccgcgagctctaatacgactcactatagggcgtcgactcgatcaatggaagatgaggcattgccgaagaaaagattaatggatttgaacacacagcaacagaaactacatgaagtgaaacacaggaaaaaaaagataaagaaacgaaaagaaaagggcatcagtgagcttcagcagaagttccatcggccttacatatgtgtaagcagaggccctgtaggagcagaggcagggggaaaatactttaagaaataatgtctaaaagtttttcaaatatgaggaaaaacataaaaccacagatccaagaagctcaacaaaacaaagcacaagaaacaggaagaaattaaaagttatatcacagtcaaattgctgaaaaccagcaacaaagagaatatcttaagagtatcagaggaaaagagattaatgacaggccaagaaacaatgaaaacaatacagatttcttgtaggaaacacaagacaaaagacattttttaaaaccaaaaggaaaaaaaatgctacattaaaatgttttttacccactgaaagtatatttcaaaacatattttaggccaggcttggtggctcacacctgtaatcccagcactttgggaggccaaggtgggtggatcgcttaaggtcaggagttcgagaccagcctggccaatatagcgaaaccccatctgtactaaaaacacaaaaattagctgggtgtggtgacacatgcctgtaatcccaggtactcaggaggctaaggcaggagaattgcttgaactgggaggcagaggtggtgagccaagattgcaccagtgcactccagccttggtgacagagtgaaactccatctcaaaaacaaacaaacaaaatacatatacataaatatatatgcacatatatatacatatataaatatatatacacatatataaatctatatacatatatacatatatacacatatataaatctatatacatatatatacatatataatatatttacatatataaatatatacatatataaatatacatatataaatacatatataaatatacatatataaatatacatatataaatatacatatataaatatatacatatataaatatacatatataaatatatatacatatataaatatataaatatacaagtatatacaaatatatacatatataaatgtatatacgtatatacatatatatataaatatataaaaaaacttttggctgggcacctttccaaatctcatggcacatataagtctcatggtaacctcaaataaaaaaacatataacagatacaccaaaaataaaaaccaataaattaaatcatgccaccagaagaaattaccttcactaaaaggaacacaggaaggaaagaaagaaggaagagaagaccatgaaacaaccagaaaacaaacaacaaaacagcaggagtaattcctgacttatcaataataatgctgggtgtaaatggactaaactctccaatcaaaagacatagagtggctgaatggacgaaaaaaacaagactcaataatctgttgcctacaagaatatacttcacctataaagggacacatagactgaaaataaaaggaaggaaaaatattctatgcaaatggaaaccaaaaaaagaacagaactagctacacttatatcagacaaaatagatttcaagacaaaaagtacaaaaagagacaaagtaattatataataataaagcaaaaagatataacaattgtgaatttatatgcgcccaacactgggacacccagatatatacagcaaatattattagaactaaggagagagagagatccccatacaataatagctggagacttcaccccgcttttagcattggacagatcatccagacagaaaatcaaccaaaaaattggacttaatctataatatagaacaaatgtacctaattgatgtttacaagacatttcatccagtagttgcagaatatgcattttttcctcagcatatggatcattctcaaggatagaccatatattaggccacagaacaagccattaaaaattcaaaaaaattgagccaggcatgatggcttatgcttgtaattacagcactttggggagggtgaggtgggaggatgtcttgagtacaggagtttgagaccagcctgggcaaaatagtgagaccctgtctctacaaacttttttttttaattagccaggcatagtggtgtgtgcctgtagtcccagctacttaggaggctgaagtgggaggatcacttgagcccaagagttcaaggctacggtgagccatgattgcaacaccacacaccagccttggtgacagaatgagaccctgtctcaaaaaaaaaaaaaaaaattgaaataatataaagcatcttctctggccacagtggaacaaaaccagaaatcaacaacaagaggaattttgaaaactatacaaacacatgaaaattaaacaatatacttctgaataaccagtgagtcaatgaagaaattaaaaaggaaattgaaaaatttatttaagcaaatgataacggaaacataacctctcaaaacccacggtatacagcaaaagcagtgctaagaaggaagtttatagctataagcagctacatcaaaaaagtagaaaagccaggcgcagtggctcatgcctgtaatcccagcactttgggaggccaaggcgggcagatcgcctgaggtcaggagttcgagaccagcctgaccaacacagagaaaccttgtcgctactaaaaatacaaaattagctgggcatggtggcacatgcctgtaatcccagctactcgggaggctgaggcaggataaccgcttgaacccaggaggtggaggttgcggtgagccgggattgcgccattggactccagcctgggtaacaagagtgaaaccctgtctcaagaaaaaaaaaaaagtagaaaaacttaaaaatacaacctaatgatgcaccttaaagaactagaaaagcaagagcaaactaaacctaaaattggtaaaagaaaagaaataataaagatcagagcagaaataaatgaaactgaaagataacaatacaaaagatcaacaaaattaaaagttggttttttgaaaagataaacaaaattgacaaacctttgcccagactaagaaaaaaggaaagaagacctaaataaataaagtcagagatgaaaaaagagacattacaactgataccacagaaattcaaaggatcactagaggctactatgagcaactgtacactaataaattgaaaaacctagaaaaaatagataaattcctagatgcatacaacctaccaagattgaaccatgaagaaatccaaagcccaaacagaccaataacaataatgggattaaagccataataaaaagtctcctagcaaagagaagcccaggacccaatggcttccctgctggattttaccaatcatttaaagaagaatgaattccaatcctactcaaactattctgaaaaatagaggaaagaatacttccaaactcattctacatggccagtattaccctgattccaaaaccagacaaaaacacatcaaaaacaaacaaacaaaaaaacagaaagaaagaaaactacaggccaatatccctgatgaatactgatacaaaaatcctcaacaaaacactagcaaaccaaattaaacaacaccttcgaaagatcattcattgtgatcaagtgggatttattccagggatggaaggatggttcaacatatgcaaatcaatcaatgtgatacatcatcccaacaaaatgaagtacaaaaactatatgattatttcactttatgcagaaaaagcatttgataaaattctgcacccttcatgataaaaaccctcaaaaaaccaggtatacaagaaacatacaggccaggcacagtggctcacacctgcgatcccagcactctgggaggccaaggtgggatgattgcttgggcccaggagtttgagactagcctgggcaacaaaatgagacctggtctacaaaaaacttttttaaaaaattagccaggcatgatggcatatgcctgtagtcccagctagtctggaggctgaggtgggagaatcacttaagcctaggaggtcgaggctgcagtgagccatgaacatgtcactgtactccagcctagacaacagaacaagaccccactgaataagaagaaggagaaggagaagggagaaaggagggagaagggaggaggaggagaaggaggaggtggaggagaagtggaaggggaaggggaagggaaagaggaagaagaagaaacatatttcaacataataaaagccctatatgacagaccgaggtagtattatgaggaaaaactgaaagcctttcctctaagatctggaaaatgacaagggcccactttcaccactgtgattcaacatagtactagaagtcctagctagagcaatcagataagagaaagaaataaaaggcatccaaactggaaaggaagaagtcaaattatcctgtttgcagatgatatgatcttatatctggaaaagacttaagacaccactaaaaaactattagagctgaaatttggtacagcaggatacaaaatcaatgtacaaaaatcagtagtatttctatattccaacagcaaacaatctgaaaaagaaaccaaaaaagcagctacaaataaaattaaacagctaggaattaaccaaagaagtgaaagatctctacaatgaaaactataaaatattgataaaagaaattgaagagggcacaaaaaaagaaaagatattccatgttcatagattggaagaataaatactgttaaaatgtccatactacccaaagcaatttacaaattcaatgcaatccctattaaaatactaatgacgttcttcacagaaatagaagaaacaattctaagatttgtacagaaccacaaaagacccagaatagccaaagctatcctgaccaaaaagaacaaaactggaagcatcacattacctgacttcaaattatactacaaagctatagtaacccaaactacatggtactggcataaaaacagatgagacatggaccagaggaacagaatagagaatccagaaacaaatccatgcatctacagtgaactcatttttgacaaaggtgccaagaacatactttggggaaaagataatctcttcaataaatggtgctggaggaactggatatccatatgcaaaataacaatactagaactctgtctctcaccatatacaaaagcaaatcaaaatggatgaaaggcttaaatctaaaacctcaaactttgcaactactaaaagaaaacaccggagaaactctccaggacattggagtgggcaaagacttcttgagtaattccctgcaggcacaggcaaccaaagcaaaaacagacaaatgggatcatatcaagttaaaaagcttctgcccagcaaaggaaacaatcaacaaagagaagagacaacccacagaatgggagaatatatttgcaaactattcatctaacaaggaattaataaccagtatatataaggagctcaaactactctataagaaaaacacctaataagctgattttcaaaaataagcaaaagatctgggtagacatttctcaaaataagtcatacaaatggcaaacaggcatctgaaaatgtgctcaacaccactgatcatcagagaaatgcaaatcaaaactactatgagagatcatctcaccccagttaaaatggcttttattcaaaagacaggcaataacaaatgccagtgaggatgtggataaaaggaaacccttggacactgttggtgggaatggaaattgctaccactatggagaacagtttgaaagttcctcaaaaaactaaaaataaagctaccatacagcaatcccattgctaggtatatactccaaaaaagggaatcagtgtatcaacaagctatctccactcccacatttactgcagcactgttcatagcagccaaggtttggaagcaacctcagtgtccatcaacagacgaatggaaaaagaaaatgtggtgcacatacacaatggagtactacgcagccataaaaaagaatgagatcctgtcagttgcaacagcatggggggcactggtcagtatgttaagtgaaataagccaggcacagaaagacaaacttttcatgttctcccttacttgtgggagcaaaaattaaaacaattgacatagaaatagaggagaatggtggttctagaggggtgggggacagggtgactagagtcaacaataatttattgtatgttttaaaataactaaaagagtataattgggttgtttgtaacacaaagaaaggataaatgcttgaaggtgacagataccccatttaccctgatgtgattattacacattgtatgcctgtatcaaaatatctcatgtatgctatagatataaaccctactatattaaaaattaaaattttaatggccaggcacggtggctcatgtccataatcccagcactttgggaggccgaggcggtggatcacctgaggtcaggagtttgaaaccagtctggccaccatgatgaaaccctgtctctactaaagatacaaaaattagccaggcgtggtggcacatacctgtagtcccaactactcaggaggctgagacaggagaattgcttgaacctgggaggcggaggttgcagtgagccgagatcatgccactgcactgcagcctgggtgacagagcaagactccatctcaaaacaaaaacaaaaaaaagaagattaaaattgtaatttttatgtaccgtataaatatatactctactatattagaagttaaaaattaaaacaattataaaaggtaattaaccacttaatctaaaataagaacaatgtatgtggggtttctagcttctgaagaagtaaaagttatggccacgatggcagaaatgtgaggagggaacagtggaagttactgttgttagacgctcatactctctgtaagtgacttaattttaaccaaagacaggctgggagaagttaaagaggcattctataagccctaaaacaactgctaataatggtgaaaggtaatctctattaattaccaataattacagatatctctaaaatcgagctgcagaattggcacgtctgatcacaccgtcctctcattcacggtgctttttttcttgtgtgcttggagattttcgattgtgtgttcgtgtttggttaaacttaatctgtatgaatcctgaaacgaaaaatggtggtgatttcctccagaagaattagagtacctggcaggaagcaggtggctctgtggacctgagccacttcaatcttcaagggtctctggccaagacccaggtgcaaggcagaggcctgatgacccgaggacaggaaagctcggatgggaaggggcgatgagaagcctgcctcgttggtgagcagcgcatgaagtgcccttatttacgctttgcaaagattgctctggataccatctggaaaaggcggccagcgggaatgcaaggagtcagaagcctcctgctcaaacccaggccagcagctatggcgcccacccgggcgtgtgccagagggagaggagtcaaggcacctcgaagtatggcttaaatctttttttcacctgaagcagtgaccaaggtgtattctgagggaagcttgagttaggtgccttctttaaaacagaaagtcatggaagcacccttctcaagggaaaaccagacgcccgctctgcggtcatttacctctttcctctctccctctcttgccctcgcggtttctgatcgggacagagtgacccccgtggagcttctccgagcccgtgctgaggaccctcttgcaaagggctccacagacccccgccctggagagaggagtctgagcctggcttaataacaaactgggatgtggctgggggcggacagcgacggcgggattcaaagacttaattccatgagtaaattcaacctttccacatccgaatggatttggattttatcttaatattttcttaaatttcatcaaataacattcaggagtgcagaaatccaaaggcgtaaaacaggaactgagctatgtttgccaaggtccaaggacttaataaccatgttcagagggatttttcgccctaagtactttttattggttttcataaggtggcttagggtgcaagggaaagtacacgaggagaggactgggcggcagggctatgagcacggcaaggccaccggggagagagtccccggcctgggaggctgacagcaggaccactgaccgtcctccctgggagctgccacattgggcaacgcgaaggcggccacgctgcgtgtgactcaggaccccataccggcttcctgggcccacccacactaacccaggaagtcacggagctctgaacccgtggaaacgaacatgacccttgcctgcctgcttccctgggtgggtcaagggtaatgaagtggtgtgcaggaaatggccatgtaaattacacgactctgctgatggggaccgttccttccatcattattcatcttcacccccaaggactgaatgattccagcaacttcttcgggtgtgacaagccatgacaacactcagtacaaacaccactcttttactaggcccacagagcacggcccacacccctgatatattaagagtccaggagagatgaggctgctttcagccaccaggctggggtgacaacagcggctgaacagtctgttcctctagactagtagaccctggcaggcactcccccagattctagggcctggttgctgcttcccgagggcgccatctgccctggagactcagcctggggtgccacactgaggccagccctgtctccacaccctccgcctccaggcctcagcttctccagcagcttcctaaaccctgggtgggccgtgttccagcgctactgtctcacctgtcccactgtgtcttgtctcagcgacgtagctcgcacggttcctcctcacatggggtgtctgtctccttccccaacactcacatgcgttgaagggaggagattctgcgcctcccagactggctcctctgagcctgaacctggctcgtggcccccgatgcaggttcctggcgtccggctgcacgctgacctccatttccaggcgctccccgtctcctgtcatctgccggggcctgccggtgtgttcttctgtttctgtgctcctttccacgtccagctgcgtgtgtctctgtccgctagggtctcggggtttttataggcataggacgggggcgtggtgggccagggcgctcttgggaaatgcaacatttgggtgtgaaagtaggagtgcctgtcctcacctaggtccacgggcacaggcctggggatggagcccccgccagggacccgcccttctctgcccagcacttttctgcccccctccctctggaacacagagtggcagtttccacaagcactaagcatcctcttcccaaaagacccagcattggcacccctggacatttgccccacagccctgggaattcacgtgactacgcacatcatgtacacactcccgtccacgaccgacccccgctgttttattttaatagctacaaagcagggaaatccctgctaaaatgtcctttaacaaactggttaaacaaacgggtccatccgcacggtggacagttcctcacagtgaagaggaacatgccgtttataaagcctgcaggcatctcaagggaattacgctgagtcaaaactgccacctccatgggatacgtacgcaacatgctcaaaaagaaagaatttcaccccatggcaggggagtggttggggggttaaggacggtgggggcagcagctgggggctactgcacgcaccttttactaaagccagtttcctggttctgatggtattggctcagttatgggagactaaccataggggagtggggatgggggaacccggaggctgtgccatctttgccatgcccgagtgtcctgggcaggataatgctctagagatgcccacgtcctgattcccccaaacctgtggacagaacccgcccggccccagggcctttgcaggtgtgatctccgtgaggaccctgaggtctgggatccttcgggactacctgcaggcccgaaaagtaatccaggggttctgggaagaggcgggcaggagggtcagaggggggcagcctcaggacgatggaggcagtcagtctgaggctgaaaagggagggagggcctcgagcccaggcctgcaagcgcctccagaagctggaaaaagcggggaagggaccctccacggagcctgcagcaggaaggcacggctggcccttagcccaccagggcccatcgtggacctccggcctccgtgccataggagggcactcgcgctgcccttctagcatgaagtgtgtggggatttgcagaagcaacaggaaacccatgcactgtgaatctaggattatttcaaaacaaaggtttacagaaacatccaaggacagggctgaagtgcctccgggcaagggcagggcaggcacgagtgattttatttagctattttattttatttacttactttctgagacagagttatgctcttgttgcccaggctggagtgcagcggcatgatcttggctcactgcaacctccgtctcctgggttcaagcaattctcgtgcctcagcctcccaagtagctgggatttcaggcgtgcaccaccacacccggctaattttgtatttttagtagagatgggctttcaccatgttggtcaggctgatctcaaaatcctgacctcaggtgatccgcccacctcagcctcccaaagtgctgggattacaggcatgagccactgcacctggcctatttaaccattttaaaacttccctgggctcaagtcacacccactggtaaggagttcatggagttcaatttcccctttactcaggagttaccctcctttgatattttctgtaattcttcgtagactggggatacaccgtctcttgacatattcacagtttctgtgaccacctgttatcccatgggacccactgcaggggcagctgggaggctgcaggcttcaggtcccagtggggttgccatctgccagtagaaacctgatgtagaatcagggcgcgagtgtggacactgtcctgaatctcaatgtctcagtgtgtgctgaaacatgtagaaattaaagtccatccctcctactctactgggattgagccccttccctatccccccccaggggcagaggagttcctctcactcctgtggaggaaggaatgatactttgttatttttcactgctggtactgaatccactgtttcatttgttggtttgtttgttttgttttgagaggcggtttcactcttgttgctcaggctggagggagtgcaatggcgcgatcttggcttactgcagcctctgcctcccaggttcaagtgattctcctgcttccgcctcccatttggctgggattacaggcacccgccaccatgcccagctaattttttgtatttttagtagagacgggggtgggggtggggttcaccatgttggccaggctggtctcgaacttctgacctcagatgatccacctgcctctgcctcctaaagtgctgggattacaggtgtgagccaccatgcccagctcagaatttactctgtttagaaacatctgggtctgaggtaggaagctcaccccactcaagtgttgtggtgttttaagccaatgatagaatttttttattgttgttagaacactcttgatgttttacactgtgatgactaagacatcatcagcttttcaaagacacactaactgcacccataatactggggtgtcttctgggtatcagcgatcttcattgaatgccgggaggcgtttcctcgccatgcacatggtgttaattactccagcataatcttctgcttccatttcttctcttccctcttttaaaattgtgttttctatgttggcttctctgcagagaaccagtgtaagctacaacttaacttttgttggaacaaattttccaaaccgcccctttgccctagtggcagagacaattcacaaacacagccctttaaaaaggcttagggatcactaaggggatttctagaagagcgacccgtaatcctaagtatttacaagacgaggctaacctccagcgagcgtgacagcccagggagggtgcgaggcctgttcaaatgctagctccataaataaagcaatttcctccggcagtttctgaaagtaggaaaggttacatttaaggttgcgtttgttagcatttcagtgtttgccgacctcagctacagcatccctgcaaggcctcgggagacccagaagtttctcgccccttagatccaaacttgagcaacccggagtctggattcctgggaagtcctcagctgtcctgcggttgtgccggggccccaggtctggaggggaccagtggccgtgtggcttctactgctgggctggaagtcgggcctcctagctctgcagtccgaggcttggagccaggtgcctggaccccgaggctgccctccaccctgtgcgggcgggatgtgaccagatgttggcctcatctgccagacagagtgccggggcccagggtcaaggccgttgtggctggtgtgaggcgcccggtgcgcggccagcaggagcgcctggctccatttcccaccctttctcgacgggaccgccccggtgggtgattaacagatttggggtggtttgctcatggtggggacccctcgccgcctgagaacctgcaaagagaaatgacgggcctgtgtcaaggagcccaagtcgcggggaagtgttgcagggaggcactccgggaggtcccgcgtgcccgtccagggagcaatgcgtcctcgggttcgtccccagccgcgtctacgcgcctccgtcctccccttcacgtccggcattcgtggtgcccggagcccgacgccccgcgtccggacctggaggcagccctgggtctccggatcaggccagcggccaaagggtcgccgcacgcacctgttcccagggcctccacatcatggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgggcctccccggggtcggcgtccggctggggttgagggcggccggggggaaccagcgacatgcggagagcagcgcaggcgactcagggcgcttcccccgcaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggtgaggaggtggtggccgtcgagggcccaggccccagagctgaatgcagtaggggctcagaaaagggggcaggcagagccctggtcctcctgtctccatcgtcacgtgggcacacgtggcttttcgctcaggacgtcgagtggacacggtgatcgagtcgactccctttagtgagggttaattgagctcgcggccgc

SEQ ID NO: 2 - DNA, 2426 bp, Homo sapiens
gatcagttagaaagttactagtccacatataaagtgccaagtcttgtactcaagattataagcaataggaatttaaaaaaagaaattatgaaaactgacaagatttagtgcctacttagatatgaaggggaaagaagggtttgagataatgtgggatgctaagagaatggtggtagtgttgacatataactcaaagcatttagcatctactctatgtaaggtactgtgctaagtgcaatagtgctaaaaacaggagtcagattctgtccgtaaaaaactttacaacctggcagatgctatgaaagaaaaaggggatgggagagagagaaggagggagagagatggagagggagatattttacttttctttcagatcgaggaccgacagcgacaactccacggagtttatctaactgaatacgagtaaaacttttaagatcatcctgtcatttatatgtaaaactgcactatactggccattataaaaattcgcggccgggtgcggtggctcatacctgtaatcccagcactttgggaggccgaagcgggtggatcacttgagccctggcgttcgagaccagcctgggcaacatggtgaaacccccgtctctactaaaaacacaaaaactagctgggcgtggtggcaggcgcctgtaatcccagctactcaggaggctgagacacgagaatcgcttgaacccgggagcagaggttgcagtgagccgagatcacgccactagactccatccagcctgggcgaaagagcaagactccgtctcaaaaaaaaaaatcgttacaatttatggtggattactcccctctttttacctcatcaagacacagcactactttaaagcaaagtcaatgattgaaacgcctttctttcctaataaaagggagattcagtccttaagattaataatgtagtagttacacttgattaaagccatcctctgctcaaggagaggctggagaaggcattctaaggagaagggggcagggtaggaactcggacgcatcccactgagccgagacaagattctgctgtagtcagtgctgcctgggaatctattttcacaaagttctccaaaaaatgtgatgatcaaaactaggaattagtgttctgtgtcttaggccctaaaatcttcctgtgaattccatttttaaggtagtcgaggtgaaccgcgtctggtctgcagaggatagaaaaaaggccctctgatacctcaagttagtttcacctttaaagaaggtcggaagtaaagacgcaaagcctttcccggacgtgcggaagggcaacgtccttcctcatggccggaaatggaactttaatttcccgttccccccaaccagcccgcccgagagagtgactctcacgagagccgcgagagtcagcttggccaatccgtgcggtcggcggccgctccctttataagccgactcgcccggcagcgcaccgggttgcggagggtgggcctgggaggggtggtggccattttttgtctaaccctaactgagaagggcgtaggcgccgtgcttttgctccccgcgcgctgtttttctcgctgactttcagcgggcggaaaagcctcggcctgccgccttccaccgttcattctagagcaaacaaaaaatgtcagctgctggcccgttcgcccctcccggggacctgcggcgggtcgcctgcccagcccccgaaccccgcctggaggccgcggtcggcccggggcttctccggaggcacccactgccaccgcgaagagttgggctctgtcagccgcgggtctctcgggggcgagggcgaggttcaggcctttcaggccgcaggaagaggaacggagcgagtccccgcgcgcggcgcgattccctgagctgtgggacgtgcacccaggactcggctcacacatgcagttcgctttcctgttggtggggggaacgccgatcgtgcgcatccgtcacccctcgccggcagtgggggcttgtgaacccccaaacctgactgactgggccagtgtgctgcaaattggcaggagacgtgaaggcacctccaaagtcggccaaaatgaatgggcagtgagccggggttgcctggagccgttcctgcgtgggttctcccgtcttccgctttttgttgccttttatggttgtattacaacttagttcctgctctgcagattttgttgaggtttttgcttctcccaaggtagatctcgaccagtccctcaacggggtgtggggagaacagtcatttttttttgagagatcatttaacatttaatgaatatttaattagaagatctaaatgaacattggaaattgtgttcctttaatggtcatcggtttatgccagaggttagaagtttcttttttgaaaaattagaccttggcgatgaccttgagcagtaggatataacccccacaagctt